(19) United States
(12) Patent Application Publication
(10) Pub. No.: US 2023/0364117 A1
(43) Pub. Date: Nov. 16, 2023

(54) BRAIN HEALTH SUPPLEMENTS

(71) Applicant: Melaleuca, Inc., Idaho Falls, ID (US)

(72) Inventors: Nasser A. Fredj, Ammon, ID (US); Jordan Joy, Idaho Falls, ID (US); Luping Yan, Idaho Falls, ID (US)

(21) Appl. No.: 18/195,713

(22) Filed: May 10, 2023

Related U.S. Application Data
(60) Provisional application No. 63/340,367, filed on May 10, 2022.

(51) Int. Cl. A61K 31/685; A61K 36/16; A61K 36/68; A61K 45/06; A61K 9/28

(52) U.S. Cl. CPC A61K 31/685; A61K 36/16; A61K 36/68; A61K 45/06; A61K 9/2813; A61K 9/2866; A61K 9/282

(57) ABSTRACT

Brain health supplement compositions are described herein. For example, this document provides brain health supplement compositions containing phosphatidylserine, a Ginkgo biloba extract, and a Bacopa monnieri extract.

### CROSS-REFERENCE TO RELATED APPLICATIONS

This application claims priority from U.S. Provisional Application Ser. No. 63/340,367, filed May 10, 2022. The disclosure of the prior application is considered part of (and is incorporated by reference in) the disclosure of this application.

### TECHNICAL FIELD

This document relates to the field of brain health supplements. For example, this document relates to brain health supplement compositions useful for increasing human or animal memory recall.

### BACKGROUND

Many people experience memory-related problems that compromise their overall quality of life. The pharmaceutical approaches developed to treat memory-related issues to date have been lacking. More than two hundred promising drug candidates for dementia have failed clinical trials in the past decade, suggesting that memory-related problems and dementia, in particular, may be highly complex. Moreover, few, if any, dietary supplements are available for treating memory-related problems.

### SUMMARY

This document provides brain health supplement compositions. For example, this document provides brain health supplement compositions useful for human or animal consumption. The brain health supplement compositions provided herein can improve memory recall. In general, the brain health supplement compositions provided herein include phosphatidylserine, a Ginkgo biloba extract, and a Bacopa monnieri extract.

In general, the brain health supplement compositions described herein can improve brain health, and memory recall, memory retention, and mental sharpness (e.g., by providing neurotransmission support and improving nerve health) of a subject that was administered the composition. As provided herein, the brain health supplement compositions can include one or more of the following benefits. The combination of ingredients in the compositions, including phosphatidylserine, a Ginkgo biloba extract, and a Bacopa monnieri extract, offers diverse and complimentary mechanisms of action for the subject's general brain health and memory. For example, the brain health supplement compositions provided herein can act through neuroprotective antioxidant mechanisms and/or can alter different neurotransmitters (e.g., serotonin, 5-hydroxytryptamine, dopamine, acetylcholine, and gamma aminobutyric acid) to provide cognitive benefits. In some cases, the brain health supplement compositions also can act to boost cholinergic function (e.g., by increasing acetylcholine release), thereby providing cognitive benefits. In addition, the brain health supplement compositions provided herein can have an antioxidant effect, inhibit beta-amyloid peptide aggregation, and/or decrease expression of peripheral benzodiazepine receptor for stress alleviation. In some cases, the combination of phosphatidylserine, a Ginkgo biloba extract, and a Bacopa monnieri extract in the composition can provide synergistic effects, resulting in brain health benefits at a level that is greater than the level that would be attained using phosphatidylserine, the Ginkgo biloba extract, or the Bacopa monnieri extract alone, or greater than the level that would be attained using two of the three ingredients without the third.

In general, one aspect of this disclosure features a composition including, consisting of, or consisting essentially of phosphatidylserine, a Ginkgo biloba extract, and a Bacopa monnieri extract, where the composition is formulated as a tablet, a capsule, or a soft gel. The composition can include about 8 wt % to about 14 wt % of the phosphatidylserine, about 9 wt % to about 20 wt % of the Ginkgo biloba extract, and about 11 wt % to about 23 wt % of the Bacopa monnieri extract. The composition can include about 8 wt % to about 10 wt % of the phosphatidylserine, about 11 wt % to about 17 wt % of the Ginkgo biloba extract, and about 13 wt % to about 19 wt % of the Bacopa monnieri extract. The composition can include about 8.75 wt % of the phosphatidylserine, about 14 wt % of the Ginkgo biloba extract, and about 16 wt % of the Bacopa monnieri extract. In some cases, one tablet, capsule, or soft gel includes a full serving of the composition. In some cases, two tablets in combination, two capsules in combination, or two soft gels in combination include a full serving of the composition. In some cases, three tablets in combination, three capsules in combination, or three soft gels in combination include a full serving of the composition. The full serving of the composition can include about 25 mg to about 1200 mg of the phosphatidylserine, about 40 mg to about 400 mg of the Ginkgo biloba extract, and about 100 mg to about 800 mg of the Bacopa monnieri extract.

The composition can further include one or more of calcium carbonate powder, microcrystalline cellulose (MCC, such as MCC 101), croscarmellose sodium, stearic acid, silicon dioxide, and magnesium stearate. The composition can include about 25 wt % to about 37 wt % of the calcium carbonate powder, about 7 wt % to about 10 wt % of the MCC, about 3.5 wt % to about 5.5 wt % of the croscarmellose sodium, about 1 wt % to about 2 wt % of the stearic acid, about 1 wt % to about 2 wt % of the silicon dioxide, and about 0.5 wt % to about 1 wt % of the magnesium stearate. The full serving of the composition can include about 350 mg to about 450 mg of the calcium carbonate powder, about 50 mg to about 150 mg of the MCC, about 45 mg to about 75 mg of the croscarmellose sodium, about 17.5 mg to about 30 mg of the stearic acid, about 20 mg to about 30 mg of the silicon dioxide, and about 10 mg to about 15 mg of the magnesium stearate.

The tablet, capsule, or soft gel can include a coating, where the coating includes cellulose, calcium carbonate powder, and carnauba wax. The composition can include about 1.6 wt % to about 2.4 wt % of the cellulose, about 0.5 wt % to about 1.3 wt % of the calcium carbonate powder in the coating, and about 0.008 wt % to about 0.012 wt % of the carnauba wax. The composition can include about 1.65 wt % of the cellulose, about 0.6 wt % of the calcium carbonate powder in the coating, and about 0.01 wt % of the carnauba wax.

In some cases, the tablet, capsule, or soft gel dissolves in water in less than 60 minutes. In some cases, the tablet, capsule, or soft gel does not dissolve in water in less than 60 minutes. In some cases, the tablet, capsule, or soft gel comprises a pH-dependent coating thereover. In some cases, the pH-dependent coating is insoluble at a pH of less than about 5, less than about 4.5, less than about 4, or less than about 3.5. In some cases, the pH-dependent coating dissolves at a pH of about 5 or more, about 5.5 or more, about 6 or more, about 6.5 or more, or about 7 or more. In some cases, the pH-dependent coating comprises one or more of methacrylic acid copolymer, cellulose acetate phthalate, hydroxypropyl methylcellulose phthalate, hydroxypropyl methylcellulose acetate succinate, polyvinyl acetate phthalate, cellulose acetate trimellitate, carboxymethylethylcellulose, shellac, and zein.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DETAILED DESCRIPTION

This document provides brain health supplement compositions that contain phosphatidylserine, a Ginkgo biloba extract, and a Bacopa monnieri extract. For example, a brain health supplement composition provided herein can include about 8 wt % to about 14 wt % of the phosphatidylserine, about 9 wt % to about 20 wt % of the Ginkgo biloba extract, and about 11 wt % to about 23 wt % of the Bacopa monnieri extract. A brain health supplement composition can be in the form of a liquid, solution, suspension, tablet, powder, cream, mist, atomized vapor, aerosol, soft gelatin capsule, hard gelatin capsule, a gel, a confectionary, a shake, a bar, and a supplemented food. For example, a brain health supplement composition can be formulated as a tablet, a capsule, or a soft gel.

As described herein, the brain health supplement compositions provided herein contain phosphatidylserine. The phosphatidylserine can be obtained using any appropriate process, and can be from any appropriate source. In some cases, phosphatidylserine can be obtained from a sunflower seed extract (e.g., Helianthus annuus extract). In some cases, phosphatidylserine can be obtained commercially. A brain health supplement composition provided herein can contain any appropriate amount of phosphatidylserine. For example, at least 5 weight percent (e.g., at least 5, 6, 7, 8, 9, 10, 11, 12, 13, 14, 15, 20, or 25 weight percent) of a brain health supplement composition provided herein can be phosphatidylserine. In some cases, a brain health supplement composition can include from about 5 wt % to about 20 wt % of phosphatidylserine. For example, a brain health supplement composition can include from about 5 wt % to about 18 wt % of phosphatidylserine, from about 5 wt % to about 15 wt % of phosphatidylserine, about 8 wt % to about 14 wt %, from about 7 wt % to about 12 wt % of phosphatidylserine, from about 8 wt % to about 10 wt % of phosphatidylserine, or from about 8.75 wt % of phosphatidylserine.

In some cases, a brain health supplement composition provided herein can include about 25 mg to about 1500 mg (e.g., about 25 mg to about 1200 mg, about 25 mg to about 1000 mg, about 50 mg to about 500 mg, about 50 mg to about 250 mg, about 50 mg to about 200 mg, about 50 mg to about 150 mg, or about 75 mg to about 125 mg) of phosphatidylserine. In some cases, a brain health supplement composition provided herein can be formulated to include an amount of phosphatidylserine such that a daily dose of about 25 mg to about 1500 mg (e.g., about 25 mg to about 1200 mg, about 25 mg to about 1000 mg, about 50 mg to about 500 mg, about 50 mg to about 250 mg, about 50 mg to about 200 mg, about 50 mg to about 150 mg, or about 75 mg to about 125 mg) of phosphatidylserine can be conveniently administered. In some cases, a brain health supplement composition provided herein can be formulated to include an amount of phosphatidylserine such that a daily dose is about 25 mg to about 1200 mg (e.g., about 75 mg to about 125 mg). The phosphatidylserine can be provided in any suitable form. For example, the phosphatidylserine can be provided as a liquid or gel, where the phosphatidylserine is present in the liquid or gel in an amount of about 70 wt %. When a brain health supplement composition contains phosphatidylserine in an amount of about 100 mg, where the phosphatidylserine is provided as a 70 wt % solution, the amount of phosphatidylserine in the brain health supplement is about 100 mg and the total weight of the solution is about 143 mg.

The brain health supplement compositions provided herein also contain a Bacopa monnieri extract. The Bacopa monnieri extract can be obtained using any appropriate method, including methods described elsewhere (see, e.g., PCT Publication No. WO2006097043). In some cases, a Bacopa monnieri extract can be obtained commercially. The brain health supplement compositions provided herein can include any appropriate amount of a Bacopa monnieri extract. For example, at least 8 weight percent (e.g., at least 8, 9, 10, 11, 12, 13, 14, 15, 20, 25, or 30 weight percent) of a brain health supplement composition provided herein can be a Bacopa monnieri extract. In some cases, a brain health supplement composition can include about 10 wt % to about 30 wt % of the Bacopa monnieri extract. For example, a brain health supplement composition can include about 10 wt % to about 30 wt % of a Bacopa monnieri extract, about 10 wt % to about 25 wt % of a Bacopa monnieri extract, about 11 wt % to about 23 wt % of a Bacopa monnieri extract, about 13 wt % to about 19 wt % of a Bacopa monnieri extract, or about 16 wt % of a Bacopa monnieri extract.

In some cases, a brain health supplement composition provided herein can include about 25 mg to about 800 mg (e.g., about 100 mg to about 800 mg, about 150 mg to about 600 mg, about 200 mg to about 600 mg, about 200 mg to about 500 mg, about 100 mg to about 450 mg, about 100 mg to about 400 mg, about 200 mg to about 400 mg, or about 250 mg to about 350 mg) of a Bacopa monnieri extract. In some cases, a brain health supplement composition provided herein can be formulated to include an amount of a Bacopa monnieri extract such that a daily dose from about 25 mg to about 800 mg (e.g., about 100 mg to about 800 mg, about 150 mg to about 600 mg, about 200 mg to about 600 mg, about 200 mg to about 600 mg, about 100 mg to about 450 mg, about 100 mg to about 400 mg, about 200 mg to about 400 mg, or about 250 mg to about 350 mg) of the Bacopa monnieri extract can be conveniently administered. In some cases, a brain health supplement composition provided herein can be formulated to include an amount of a Bacopa monnieri extract such that a daily dose is about 100 mg to about 800 mg, or about 250 mg to about 350 mg.

In some cases, the Bacopa monnieri extract can include at least 10 wt % (e.g., 10, 20, 30, 35, 40, 45, 50, 55, 60, or 70 percent) Bacopasides (e.g., one or more of Bacoposide I, II, III, IV, V, VI, VII, VIII, IX, X, and XI). In some cases, the Bacopa monnieri extract can include at least 30 wt % Bacopasides (e.g., about 40 wt % Bacopasides, about 45 wt % Bacopasides, about 50 wt % Bacopasides, about 55 wt % Bacopasides, or about 60 wt % Bacopasides).

The brain health supplement compositions provided herein also contain a Ginkgo biloba extract. The Ginkgo biloba extract can be obtained using any appropriate process, and can be from any appropriate source. In some cases, the Ginkgo biloba extract can be obtained from a Ginkgo biloba leaf. In some cases, a Ginkgo biloba extract can be obtained commercially. For example, a Ginkgo biloba extract can be obtained from Dongling Health Food Co., Ltd. (Zhejiang, PRC). The brain health supplement compositions provided herein can include any appropriate amount of a Ginkgo biloba extract. For example, at least 5 weight percent (e.g., at least 5, 6, 7, 8, 9, 10, 11, 12, 13, 14, 15, 20, 25, 30, or 40 weight percent) of a brain health supplement composition provided herein can be a Ginkgo biloba extract. In some cases, a brain health supplement composition can include about 5 wt % to about 30 wt % of a Ginkgo biloba extract. For example, a brain health supplement composition can include about 5 wt % to about 25 wt % of a Ginkgo biloba extract, about 5 wt % to about 20 wt % of a Ginkgo biloba extract, about 9 wt % to about 20 wt % of a Ginkgo biloba extract, about 11 wt % to 17 wt % of a Ginkgo biloba extract, or about 14 wt % of the Ginkgo biloba extract.

In some cases, a brain health supplement composition provided herein can include about 40 mg to about 800 mg (e.g., about 40 mg to about 600 mg, about 40 mg to about 400 mg, about 120 mg to about 400 mg, about 150 mg to about 400 mg, about 200 mg to about 400 mg, about 200 mg to about 350 mg, about 200 mg to about 300 mg, or about 225 mg to about 275 mg) of a Ginkgo biloba extract. In some cases, a brain health supplement composition provided herein can be formulated to include an amount of a Ginkgo biloba extract such that a daily dose of about 40 mg and about 800 mg (e.g., about 40 mg to about 600 mg, about 40 mg to about 400 mg, about 40 mg to about 400 mg, about 150 mg to about 400 mg, about 200 mg to about 400 mg, about 200 mg to about 350 mg, about 200 mg to about 300 mg, or about 225 mg to about 275 mg) of the Ginkgo biloba extract can be conveniently administered. For example, a brain health supplement composition provided herein can be formulated to include an amount of a Ginkgo biloba extract such that the daily dose is about 120 mg to about 400 mg (e.g., about 200 mg to about 300 mg).

The Ginkgo biloba extract can include one or more flavone glycosides (e.g., quercetin, kaempferol, and isorhamnetin). The Ginkgo biloba extract can include one or more terpene lactones (e.g., ginkgolide A, ginkgolide B, ginkgolide C, bilobalide, and other ginkgolides). In some cases, the Ginkgo biloba extract can include one or more terpene lactones and one or more flavone glycosides. The Ginkgo biloba extract can include one or more flavone glycosides in an amount of at least about 10 wt % (e.g., 10 wt %, 20 wt %, 30 wt %, 35 wt %, 40 wt %, 45 wt %, 50 wt %, 10 to 20 wt %, 20 to 30 wt %, 30 to 40 wt %, or 40 to 50 wt %). In some cases, the Ginkgo biloba extract can include one or more terpene lactones in an amount of at least about 1 wt % (e.g., 1 wt %, 2 wt %, 3 wt %, 4 wt %, 5 wt %, 6 wt %, 7 wt %, 8 wt %, 9 wt %, 10 wt %, 15 wt % 1 to 5 wt %, 5 to 10 wt %, or 10 to 15 wt %). In some cases, the Ginkgo biloba extract can include at least about 10 wt % of one or more flavone glycosides and at least about 1 wt % of one or more terpene lactones. For example, the Ginkgo biloba extract can include at least about 20 wt % of one or more flavone glycosides and at least about 4 wt % of one or more terpene lactones. In some cases, the Ginkgo biloba extract can include about 24% wt % of one or more flavone glycosides and about 6 wt % of one or more terpene lactones.

In some cases, the brain health supplement provided herein can include phosphatidylserine, a Ginkgo biloba extract, and a Bacopa monnieri extract, where the composition is formulated as a tablet, a capsule, or a soft gel. In some cases, the brain health supplement provided herein can include about 8 wt % to about 14 wt % of the phosphatidylserine, about 9 wt % to about 20 wt % of the Ginkgo biloba extract, and about 11 wt % to about 23 wt % of the Bacopa monnieri extract. In some cases, the brain health supplement provided herein can include about 8 wt % to about 10 wt % of the phosphatidylserine, about 11 wt % to about 17 wt % of the Ginkgo biloba extract, and about 13 wt % to about 19 wt % of the Bacopa monnieri extract. In some cases, the brain health supplement provided herein can include about 8.25 wt % of the phosphatidylserine, about 14 wt % of the Ginkgo biloba extract, and about 16 wt % of the Bacopa monnieri extract.

The brain health supplement compositions provided herein can be formulated such that a daily dose includes about 25 mg to about 1200 mg of the phosphatidylserine, about 40 mg to about 400 mg of the Ginkgo biloba extract, and about 100 mg to about 800 mg of the Bacopa monnieri extract. For example, a brain health supplement composition provided herein can be formulated such that a daily dose includes about 50 mg to about 500 mg of the phosphatidylserine, about 150 mg to about 400 mg of the Ginkgo biloba extract, and about 150 mg to about 450 mg of the Bacopa monnieri extract, or about 50 mg to about 200 mg of the phosphatidylserine, about 200 mg to about 300 mg of the Ginkgo biloba extract, and about 200 mg to about 350 mg of the Bacopa monnieri extract.

The brain health supplement composition provided herein can be formulated to include one or more additional active ingredients. For example, the brain health supplement composition can include phosphatidylserine, a Ginkgo biloba extract, a Bacopa monnieri extract, and one or more additional active ingredients, such as one or more of acetyl-L-carnitine, acetyl CoA, vitamin B12, huperzine A, vitamin E, pyridoxine, and the like. In some cases, the additional active ingredients can include one or more acetylcholinesterase inhibitors. Examples of acetylcholinesterase inhibitors that can be included within a brain health supplement composition provided herein include, without limitation, huperzine A, carbamates (e.g., physostigmine, neostigmine, pyridostigmine, ambenonium, demarcarium, and rivastigmine), caffeine, piperidines (e.g., donepezil), xanthostigmine, aminobenzoic acid, flavonoids, pyrrolo-isoxazole, edrophonium, ladostigil, ungeremine, lactucopicrin, coumarin, donepezil, galantamine, rivastigmine, and tacrine. Acetylcholinesterase inhibitors can be obtained, for example, as described elsewhere (see, e.g., Chinese Patent No. CN103951618).

In some cases, a brain health supplement composition provided herein can include one or more of the following additional active ingredients: carotenoids (e.g., alpha-carotene, beta-carotene, lycopene, lutein, zeaxanthin, and/or cryptoxanthin), phenolic compounds (e.g., flavonoids, flavonols, flavanones, catechins, anthocyanins, isoflavones, dihydroflavonols, and/or chalcones), phenolic acids (e.g., ellagic acid, tannic acid, and/or vanillin), hydroxycinnamic acid derivatives (e.g., caffeic acid, chlorogenic acid, ferulic acid, curcumin, and/or coumarins), lignans, allyl sulphides from onion or garlic, and essential oils (e.g., melaleuca oil, clove oil, cinnamon bark oil, thyme oil, oregano oil, mountain savory oil, cistus oil, Eucalyptus globulus oil, orange oil, lemongrass oil, helichrysum oil, ravensara oil, lemon oil, spearmint oil, and/or lavender oil).

In some cases, a brain health supplement composition provided herein can further include one or more of vitamin E, tocopherol, tocotrienol, vitamin A, carotene, lutein, astaxanthin, CoQ10, vitamin C, folate, uric acid, vitamin B12, and folic acid.

The brain health supplement compositions provided herein can be formulated for oral administration and can include one or more suitable excipients, flavorings, colorants, and/or other ingredients. When intended for oral use, for example, tablets, troches, lozenges, aqueous or oil suspensions, non-aqueous solutions, dispersible powders or granules (including micronized particles or nanoparticles), emulsions, hard or soft capsules, syrups or elixirs may be prepared. Compositions intended for oral use can be prepared according to any appropriate method, including standard methods for the manufacture of pharmaceutical compositions. Such compositions can contain one or more agents such as, without limitation, sweetening agents, flavoring agents, coloring agents and preserving agents, in order to provide a palatable preparation. For oral administration, tablets, capsules, or soft gels can be prepared with one or more pharmaceutically acceptable excipients, such as binding agents, fillers, lubricants, disintegrants, and/or wetting agents. In some cases, tablets can have a coating (e.g., a polymer or polysaccharide-based coating with or without plasticizers and/or pigments). Liquid preparations for oral administration can take the form of, for example, solutions, syrups, or suspension, or they can be presented as a dry product for reconstitution with saline or other suitable liquid vehicle before use. In some cases, liquid preparations can contain pharmaceutically acceptable additives such as suspending agents, emulsifying agents, non-aqueous vehicles, preservatives, buffer salts, flavoring agents, coloring agents, and sweetening agents as appropriate. Preparations for oral administration can be suitably formulated to give controlled release of one or more compounds. In some cases, tablets, capsules, or soft gels can be coated with a methacrylic acid copolymer (e.g., EUIDRAGIT® L100-55 or EUIDRAGIT® S100) for release beyond the stomach (e.g., in the intestine, colon, or both).

In some cases, the brain health supplement compositions provided herein can contain a pharmaceutically acceptable carrier for administration to a mammal. Suitable carriers include, without limitation, sterile aqueous, or non-aqueous solutions, suspensions, and emulsions. Examples of non-aqueous solvents include, without limitation, propylene glycol, polyethylene glycol, vegetable oils, and organic esters. Suitable aqueous carriers include, for example, water, alcohol, saline, and buffered solutions. Pharmaceutically acceptable carriers also can include physiologically acceptable aqueous vehicles (e.g., physiological saline) or other carriers appropriate for oral administration.

In some cases, the brain health supplement compositions provided herein can be in the form of a capsule, tablet, or soft gel, by way of an example only, configured to have a unit dosage equal to the daily desired dosage for a particular mammal. For example, if a mammal desires 100 mg of a particular agent, each tablet can include about 100 mg in weight of that agent. As used herein, mammals generally refer to humans, but also can include domesticated mammals (e.g., dogs, cats, and livestock such as cows, horses, pigs, or sheep). The dosages of a particular brain health supplement compositions provided herein will depend on many factors, including the general health of a mammal. In some cases, a total daily dose (e.g., a full serving) may be prepared and administered in the form of one or more dosage units (e.g., one tablet, capsule, or soft gel, two tablets, capsules, or soft gels, three tablets, capsules, or soft gels, four tablets, capsules or soft gels, five tablets, capsules, or soft gels, or six tablets, capsules, or soft gels). For instance, in some cases, one tablet, one capsule, or one soft gel can include a full serving (e.g., daily dosage) of the composition. In some cases, two tablets, two capsules, or two soft gels in combination can include a full serving (e.g., daily dosage) of the composition. In some cases, three tablets, three capsules, or three soft gels in combination can include a full serving (e.g., daily dosage) of the composition.

In some cases, when the brain health supplement compositions provided herein is in the form of a capsule, tablet, or soft gel, the composition can further include one or more pharmaceutically acceptable excipients, such as calcium powder (e.g., calcium carbonate powder), microcrystalline cellulose (MCC) (e.g., MCC 101), croscarmellose sodium, stearic acid, silicon dioxide (e.g., SYLOID® silicon dioxide), and magnesium stearate. In some cases, the capsule, tablet, or soft gel form of the brain health supplement compositions provided herein further includes calcium powder (e.g., calcium carbonate powder), MCC (e.g., MCC 101), croscarmellose sodium, stearic acid, silicon dioxide (e.g., SYLOID® silicon dioxide), and magnesium stearate. In some cases, the capsule, tablet, or soft gel form of the brain health supplement compositions provided herein further includes one or more of: about 10 wt % to about 50 wt % of calcium powder (e.g., calcium carbonate powder), about 5 wt % to about 15 wt % of MCC (e.g., MCC 101), about 1 wt % to about 10 wt % of croscarmellose sodium, about 0.1 wt % to about 3 wt % of stearic acid, about 0.1 wt % to about 3 wt % silicon dioxide (e.g., SYLOID® silicon dioxide), and about 0.05 wt % to about 2 wt % magnesium stearate. In some cases, the capsule, tablet, or soft gel form of the brain health supplement compositions provided herein further includes one or more of: about 25 wt % to about 37 wt % of calcium powder (e.g., calcium carbonate powder), about 7 wt % to about 10 wt % of MCC (e.g., MCC 101), about 3.5 wt % to about 5.5 wt % of croscarmellose sodium, about 1 wt % to about 2 wt % of stearic acid, about 1 wt % to about 2 wt % silicon dioxide (e.g., SYLOID® silicon dioxide), and about 0.5 wt % to about 1 wt % magnesium stearate.

In some cases, a full serving of the brain health supplement composition provided herein in the form of one or more tablets, one or more capsules, or one or more soft gels includes about 300 mg to about 500 mg of the calcium carbonate powder, about 50 mg to about 200 mg of the MCC (e.g., MCC 101), about 25 mg to about 100 mg of the croscarmellose sodium, about 10 mg to about 50 mg of the stearic acid, about 10 mg to about 50 mg of the silicon dioxide (e.g., SYLOID® silicon dioxide), and about 1 mg to about 25 mg of the magnesium stearate. In some cases, a full serving of the brain health supplement composition provided herein in the form of one or more tablets, one or more capsules, or one or more soft gels can include about 350 mg to about 450 mg of the calcium carbonate powder, about 50 mg to about 150 mg of the MCC (e.g., MCC 101), about 45 mg to about 75 mg of the croscarmellose sodium, about 17.5 mg to about 30 mg of the stearic acid, about 20 mg to about 30 mg of the silicon dioxide (e.g., SYLOID® silicon dioxide), and about 10 mg to about 15 mg of the magnesium stearate.

In some cases, a brain health supplement composition in the form of a tablet, capsule, or soft gel can be coated with a suitable coating. For example, the coating can include one or more of cellulose, calcium carbonate powder, and carnauba wax. In some cases, the coating can include cellulose, calcium carbonate powder, and carnauba wax. In some cases, the brain health supplement composition in the form of a tablet, capsule, or soft gel includes a coating in an amount of about 1 wt % to about 5 wt %, based on the total weight of the composition. In some cases, the brain health supplement composition includes about 1 wt % to about 3 wt % of cellulose, about 0.1 wt % to about 1.5 wt % of calcium carbonate powder in the coating, and about 0.001 wt % to about 0.05 wt % of canauba wax. In some cases, the brain health supplement composition includes about 1.6 wt % to about 2.4 wt % of cellulose, about 0.5 wt % to about 1.3 wt % of calcium carbonate powder in the coating, and about 0.008 wt % to about 0.012 wt % of canauba wax. In some cases, the brain health supplement composition includes about 2.2 wt % of cellulose, about 0.8 wt % of calcium carbonate powder in the coating, and about 0.01 wt % of canauba wax.

In some cases, the brain health supplement composition in the form of a tablet, capsule, or soft gel can be formulated to release the active ingredients (e.g., phosphatidylserine, a Ginkgo biloba extract, and a Bacopa monnieri extract) in water in less than 60 minutes (e.g., an immediate release formulation) at a temperature of about 37° C. The brain health supplement composition provided herein can release the active ingredients in water in less than 45 minutes at a temperature of about 37° C. In some cases, the tablet, capsule, or soft gel is formulated with a coating that will dissolve at the pH of gastric fluid. In some cases, the coating of the tablet, capsule, or soft gel exhibits an in vitro dissolution profile in simulated gastric fluid comprising at least about 95% absorption window active ingredients release after 1 hour.

In some cases, the brain health supplement composition in the form of a tablet, capsule, or soft gel is formulated to be a controlled release formulation. The controlled release formulation can include a pH-dependent coating over the tablet, capsule, or soft gel from which the release of the active ingredients depends on the pH of the surrounding environment. The pH-dependent coating material may be dispersed or dissolved in either water or in suitable organic solvents. The pH-dependent coating exhibits pH-dependent solubility such that it is insoluble in acid medium (e.g., acidic gastric fluid) but soluble in intestinal fluids (e.g., small intestine or the colon). In some cases, the pH-dependent coating, upon exposure to the pH environment of the small intestine, will cause degradation of the coating and release of the active ingredients. In some cases, the pH-dependent coating, upon exposure to the pH environment of the small intestine, will not cause significant degradation of the coating and will not release the active ingredients. In some cases, the pH-dependent coating, upon exposure to the pH environment of the colon, will cause degradation of the coating and release of the active ingredients. In some cases, the pH-dependent coating layer delays the release of the absorption window of the active ingredients until the composition passes through the stomach and reaches the higher pH environment of the small intestine. In some cases, the pH-dependent coating will dissolve at a pH of about 5, about 5.5, about 6, or about 6.5, or the pH-dependent coating will dissolve at a pH of about 5.5, about 6.0, about 6.5, or about 7.

The pH-dependent coating can include one or more enteric coating polymers. Typical examples include one or more, separately or in combination, of the following: solutions or dispersions of methacrylic acid copolymers, cellulose acetate phthalate, hydroxypropyl methylcellulose phthalate, hydroxypropyl methylcellulose acetate succinate, polyvinyl acetate phthalate, cellulose acetate trimellitate, carboxymethylethylcellulose, shellac, zein or other suitable enteric coating polymer(s). Specific enteric polymers that may be used are EUDRAGIT® L and/or EUDRAGIT® S, which preferentially dissolve in the more alkaline pH of the intestine, or polymers which dissolve slowly, e.g. a predetermined rate in the digestive tract, such as EUDRAGIT® RL (e.g. EUDRAGIT® RL 100), EUIDRAGIT® RS (e.g. EUIDRAGIT® R100), and/or blends of such EUIDRAGIT® polymers.

### Examplary Embodiments

Embodiment 1 is a composition comprising phosphatidylserine, a Ginkgo biloba extract, and a Bacopa monnieri extract, wherein the composition is formulated as a tablet, a capsule, or a soft gel.

Embodiment 2 is the composition of embodiment 1, wherein the composition comprises: about 8 wt % to about 14 wt % of the phosphatidylserine, about 9 wt % to about 20 wt % of the Ginkgo biloba extract, and about 11 wt % to about 23 wt % of the Bacopa monnieri extract.

Embodiment 3 is the composition of embodiment 1, wherein the composition comprises: about 8 wt % to about 10 wt % of the phosphatidylserine, about 11 wt % to about 17 wt % of the Ginkgo biloba extract, and about 13 wt % to about 19 wt % of the Bacopa monnieri extract.

Embodiment 4 is the composition of embodiment 1, wherein the composition comprises: about 8.75 wt % of the phosphatidylserine, about 14 wt % of the Ginkgo biloba extract, and about 16 wt % of the Bacopa monnieri extract.

Embodiment 5 is the composition of any one of embodiments 1 to 4, wherein one tablet, capsule, or soft gel comprises a full serving of the composition.

Embodiment 6 is the composition of any one of embodiments 1 to 4, wherein two tablets in combination, two capsules in combination, or two soft gels in combination comprise a full serving of the composition.

Embodiment 7 is the composition of any one of embodiments 1 to 4, wherein three tablets in combination, three capsules in combination, or three soft gels in combination comprise a full serving of the composition.

Embodiment 8 is the composition of any one of embodiments 5 to 7, wherein the full serving of the composition comprises: about 25 mg to about 1200 mg of the phosphatidylserine, about 40 mg to about 400 mg of the Ginkgo biloba extract, and about 100 mg to about 800 mg of the Bacopa monnieri extract.

Embodiment 9 is the composition of any one of embodiments 1 to 8, further comprising one or more additional active ingredients.

Embodiment 10 is the composition of embodiment 9, wherein the additional active ingredients comprise one or more of L-carnitine, vitamin B12, huperzine A, vitamin E, and pyridoxine.

Embodiment 11 is the composition of any one of embodiments 1 to 10, further comprising: calcium carbonate powder, microcrystalline cellulose (MCC), croscarmellose sodium, stearic acid, silicon dioxide, and magnesium stearate.

Embodiment 12 is the composition of embodiment 11, wherein said composition comprises: about 25 wt % to about 37 wt % of the calcium carbonate powder, about 7 wt % to about 10 wt % of the MCC, about 3.5 wt % to about 5.5 wt % of the croscarmellose sodium, about 1 wt % to about 2 wt % of the stearic acid, about 1 wt % to about 2 wt % of the silicon dioxide, and about 0.5 wt % to about 1 wt % of the magnesium stearate.

Embodiment 13 is the composition of embodiment 11 or embodiment 12, wherein one tablet, capsule, or soft gel comprises a full serving of the composition.

Embodiment 14 is the composition of any one of embodiments 11 to 13, wherein two tablets in combination, two capsules in combination, or two soft gels in combination comprise a full serving of the composition.

Embodiment 15 is the composition of any one of embodiments 11 to 13, wherein three tablets in combination, three capsules in combination, or three soft gels in combination comprise a full serving of the composition.

Embodiment 16 is the composition of any one of embodiments 13 to 15, wherein the full serving of the composition comprises: about 350 mg to about 450 mg of the calcium carbonate powder, about 50 mg to about 150 mg of the MCC, about 45 mg to about 75 mg of the croscarmellose sodium, about 17.5 mg to about 30 mg of the stearic acid, about 20 mg to about 30 mg of the silicon dioxide, and about 10 mg to about 15 mg of the magnesium stearate.

Embodiment 17 is the composition of any one of embodiments 1 to 16, wherein the tablet, capsule, or soft gel comprises a coating, and wherein the coating comprises: cellulose, calcium carbonate powder, and carnauba wax.

Embodiment 18 is the composition of embodiment 17, wherein the composition comprises: about 1.6 wt % to about 2.4 wt % of the cellulose, about 0.7 wt % to about 1.3 wt % of the calcium carbonate powder in the coating, and about 0.008 wt % to about 0.012 wt % of the carnauba wax.

Embodiment 19 is the composition of embodiment 17, wherein the composition comprises: about 2.2 wt % of the cellulose, about 0.8 wt % of the calcium carbonate powder in the coating, and about 0.01 wt % of the carnauba wax.

Embodiment 20 is the composition of any one of embodiments 1 to 19, wherein the tablet, capsule, or soft gel is formulated to release the phosphatidylserine, Ginkgo biloba extract, and Bacopa monnieri extract in water in less than 60 minutes at a temperature of about 37° C.

Embodiment 21 is the composition of any one of embodiments 1 to 19, wherein the tablet, capsule, or soft gel is formulated to release the phosphatidylserine, Ginkgo biloba extract, and Bacopa monnieri extract in water in 60 minutes or more at a temperature of about 37° C.

Embodiment 22 is the composition of any one of embodiments 1 to 21, wherein the tablet, capsule, or soft gel comprises a pH-dependent coating thereover.

Embodiment 23 is the composition of embodiment 22, wherein the pH-dependent coating is insoluble at a pH of less than about 5, less than about 4.5, less than about 4, or less than about 3.5.

Embodiment 24 is the composition of embodiment 22 or 23, wherein the pH-dependent coating dissolves at a pH of about 5 or more, about 5.5 or more, about 6 or more, about 6.5 or more, or about 7 or more.

Embodiment 25 is the composition of any one of embodiments 22 to 24, wherein the pH-dependent coating comprises one or more of methacrylic acid copolymer, cellulose acetate phthalate, hydroxypropyl methylcellulose phthalate, hydroxypropyl methylcellulose acetate succinate, polyvinyl acetate phthalate, cellulose acetate trimellitate, carboxymethylethylcellulose, shellac, and zein.

The invention will be further described in the following examples, which do not limit the scope of the invention described in the claims.

### EXAMPLES

### Example 1—A Brain Health Supplement Composition

A brain health supplement composition was prepared with the composition shown in Table 1 below.

TABLE 1
Brain Health Supplement.

| | |
|---|---|
| Bacopa (Bacopa monnieri) Leaf Extract (50 wt % Bacopasides) | 275 mg |
| Ginkgo (Ginkgo Biloba) Leaf Extract | 240 mg |
| Phosphatidylserine (70 wt %) | 100 mg |

### Example 2—A Brain Health Supplement Tablet

A brain health supplement composition was prepared as a tablet with the composition shown in Table 2 below. The tablet was prepared with calcium carbonate powder, microcrystalline cellulose, croscarmellose sodium, stearic acid, silicon dioxide, and magnesium stearate. The tablet was coated with a coating containing a mixture of cellulose, calcium carbonate powder, and carnauba wax.

TABLE 2
Brain Health Supplement Tablet.

| | |
|---|---|
| Bacopa (Bacopa monnieri) Leaf Extract (50 wt % Bacopasides) | 275 mg |
| Ginkgo (Ginkgo Biloba) Leaf Extract | 240 mg |
| Phosphatidylserine (70 wt %) | 100 mg |

### Example 3—A Brain Health Supplement Composition

A brain health supplement is prepared with the composition shown in Table 3 below.

TABLE 3
Brain Health Supplement Composition.

| | |
|---|---|
| Bacopa (Bacopa monnieri) Leaf Extract | 350 mg |
| Ginkgo (Ginkgo Biloba) Leaf Extract | 120 mg |
| Phosphatidylserine (70 wt %) | 150 mg |

### Example 4—A Brain Health Supplement Composition

A brain health supplement is prepared with the composition shown in Table 4 below.

TABLE 4
Brain Health Supplement Composition.

| | |
|---|---|
| Bacopa (Bacopa monnieri) Leaf Extract | 300 mg |
| Ginkgo (Ginkgo Biloba) Leaf Extract | 150 mg |
| Phosphatidylserine (50 wt %) | 125 mg |

### Example 5—A Brain Health Supplement Tablet

A brain health supplement composition is prepared as a tablet with the composition shown in Table 5 below. The tablet is prepared with calcium carbonate powder, microcrystalline cellulose, croscarmellose sodium, stearic acid, silicon dioxide, and magnesium stearate. The tablet is coated with a coating, where the coating is a mixture that includes cellulose, calcium carbonate powder, and carnauba wax.

TABLE 5
Brain Health Supplement Tablet.

| | |
|---|---|
| Bacopa (Bacopa monnieri) Leaf Extract (50 wt % Bacopasides) | 350 mg |
| Ginkgo (Ginkgo Biloba) Leaf Extract | 120 mg |
| Phosphatidylserine (70 wt %) | 150 mg |

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A composition comprising phosphatidylserine, a Ginkgo biloba extract, and a Bacopa monnieri extract, wherein the composition is formulated as a tablet, a capsule, or a soft gel.

2. The composition of claim 1, wherein the composition comprises:
about 8 wt % to about 14 wt % of the phosphatidylserine,
about 9 wt % to about 20 wt % of the Ginkgo biloba extract, and
about 11 wt % to about 23 wt % of the Bacopa monnieri extract.

3. The composition of claim 1, wherein the composition comprises:
about 8 wt % to about 10 wt % of the phosphatidylserine,
about 11 wt % to about 17 wt % of the Ginkgo biloba extract, and
about 13 wt % to about 19 wt % of the Bacopa monnieri extract.

4. The composition of claim 1, wherein the composition comprises:
about 8.75 wt % of the phosphatidylserine,
about 14 wt % of the Ginkgo biloba extract, and
about 16 wt % of the Bacopa monnieri extract.

5. The composition of claim 1, wherein one tablet, capsule, or soft gel comprises a full serving of the composition.

6. The composition of claim 1, wherein two or three tablets in combination, two or three capsules in combination, or two or three soft gels in combination comprise a full serving of the composition.

7. The composition of claim 1, wherein a full serving of the composition comprises:
about 25 mg to about 1200 mg of the phosphatidylserine,
about 40 mg to about 400 mg of the Ginkgo biloba extract, and
about 100 mg to about 800 mg of the Bacopa monnieri extract.

8. The composition of claim 1, further comprising one or more additional active ingredients, wherein the one or more additional active ingredients comprise one or more of L-carnitine, vitamin B12, huperzine A, vitamin E, and pyridoxine.

9. The composition of claim 1, further comprising:
calcium carbonate powder,
microcrystalline cellulose (MCC),
croscarmellose sodium,
stearic acid,
silicon dioxide, and
magnesium stearate.

10. The composition of claim 9, wherein said composition comprises:
about 25 wt % to about 37 wt % of the calcium carbonate powder,
about 7 wt % to about 10 wt % of the MCC,
about 3.5 wt % to about 5.5 wt % of the croscarmellose sodium,
about 1 wt % to about 2 wt % of the stearic acid,
about 1 wt % to about 2 wt % of the silicon dioxide, and
about 0.5 wt % to about 1 wt % of the magnesium stearate.

11. The composition of claim 9, wherein one tablet, capsule, or soft gel comprises a full serving of the composition.

12. The composition of claim 9, wherein two or three tablets in combination, two or three capsules in combination, or two or three soft gels in combination comprise a full serving of the composition.

13. The composition of claim 9, wherein a full serving of the composition comprises:
about 350 mg to about 450 mg of the calcium carbonate powder,
about 50 mg to about 150 mg of the MCC,
about 45 mg to about 75 mg of the croscarmellose sodium,
about 17.5 mg to about 30 mg of the stearic acid,
about 20 mg to about 30 mg of the silicon dioxide, and
about 10 mg to about 15 mg of the magnesium stearate.

14. The composition of claim 1, wherein the tablet, capsule, or soft gel comprises a coating, and wherein the coating comprises:
cellulose,
calcium carbonate powder, and
carnauba wax.

15. The composition of claim 14, wherein the composition comprises:
about 1.6 wt % to about 2.4 wt % of the cellulose,
about 0.7 wt % to about 1.3 wt % of the calcium carbonate powder in the coating, and
about 0.008 wt % to about 0.012 wt % of the carnauba wax.

16. The composition of claim 14, wherein the composition comprises:
about 2.2 wt % of the cellulose,
about 0.8 wt % of the calcium carbonate powder in the coating, and
about 0.01 wt % of the carnauba wax.

17. The composition of claim 1, wherein the tablet, capsule, or soft gel is formulated to release the phosphatidylserine, Ginkgo biloba extract, and Bacopa monnieri extract in water in less than 60 minutes at a temperature of about 37° C.

18. The composition of claim 1, wherein the tablet, capsule, or soft gel is formulated to release the phosphatidylserine, Ginkgo biloba extract, and Bacopa monnieri extract in water in 60 minutes or more at a temperature of about 37° C.

19. The composition of claim 1, wherein the tablet, capsule, or soft gel comprises a pH-dependent coating thereover, and wherein the pH-dependent coating is insoluble at a pH of less than about 5, less than about 4.5, less than about 4, or less than about 3.5.

20. The composition of claim 19, wherein the tablet, capsule, or soft gel comprises a pH-dependent coating thereover, and wherein the pH-dependent coating dissolves at a pH of about 5 or more, about 5.5 or more, about 6 or more, about 6.5 or more, or about 7 or more.

21. The composition of claim 19, wherein the pH-dependent coating comprises one or more of methacrylic acid copolymer, cellulose acetate phthalate, hydroxypropyl methylcellulose phthalate, hydroxypropyl methylcellulose acetate succinate, polyvinyl acetate phthalate, cellulose acetate trimellitate, carboxymethylethylcellulose, shellac, and zein.
